(19) United States
(12) Patent Application Publication
(10) Pub. No.: US 2015/0284763 A1
(43) Pub. Date: Oct. 8, 2015

(54) Method of Using Laser-Induced Breakdown Spectroscopy for the Identification and Classification of Bacteria

(71) Applicant: UNIVERSITY OF WINDSOR, Windsor (CA)

(72) Inventor: Steven J. Rehse, Windsor (CA)

(21) Appl. No.: 14/427,462

(22) PCT Filed: Sep. 11, 2013
(86) PCT No.: PCT/CA2013/000770

§ 371 Date: Mar. 11, 2015

Related U.S. Application Data
(60) Provisional application No. 61/743,918, filed on Sep. 14, 2012.

(51) Int. Cl. C12Q 1/04; G01N 21/71

(52) U.S. Cl. CPC C12Q 1/04; G01N 21/718; G01N 2201/06113; G01N 2201/02

(57) ABSTRACT

A pathogen detection system including a specimen support for supporting a test specimen sample to be analyzed, a coherent light source, an optical detector and an analyzer electronically coupled to the optical detection. The coherent light source is operable to direct a coherent light beam at said specimen support to break down and at least partially atomize said test specimen sample. The optical detector is positioned to detect a spectral signature of electromagnetic radiation emitted by the partial atomization of the test specimen sample. The analyzer compares the detected spectral signature to one or more predetermined spectral signatures for one or more pathogens.

### RELATED APPLICATIONS

This application claims the benefit of 35 USC §119(e) to U.S. Application Ser. No. 61/743,918, filed Sep. 14, 2012, and which is incorporated herein by reference in its entirety.

### SCOPE OF THE INVENTION

The present invention relates to an apparatus and process of sample preparation and presentation using laser-induced breakdown spectroscopy (LIBS) to identify and classify pathogens, and preferably pathogenic bacteria, very rapidly for a prepared original sample. More preferably the invention further uses the LIBS process to identify bacteria and evaluate potential treatment protocols for bacterial infections.

### BACKGROUND OF THE INVENTION

Pathogen detection is of the utmost importance primarily for health and safety reasons. According to studies, three areas of application account for over two thirds of all research in the field of pathogen detection: the food industry; water and environment quality control; and clinical diagnosis. Military-biodefense constitutes a small niche market for this technology. Because of the global demand for pathogen detection technology and testing, as of 2003 the pathogen specific testing market was expected to grow for all segments at a compounded annual growth rate (CAGB) of 4.5% with a total market value of $563 million. In the last published analysis of the industry, the annual worldwide investment in advanced biosensor R&D was estimated to be $300 US million and the total worldwide medical biosensor sales market was approximately $7 billion (CAD) and was projected to rise to over $10 billion (CAD) with the medical/health area being the largest sector. Over 50% and 22% of the biosensor sales are in North America and Europe, respectively.

The detection and identification of foodborne pathogens in this sector continue to rely on conventional time-consuming and culturing techniques. The existing test methods are completed in a microbiology laboratory and are not suitable for on-site monitoring. Pathogen detection using existing methods, such as enzyme linked immunosorbent assays (ELISA) and culture techniques for determining and quantifying pathogens in food have been well established. In terms of speed, these methods cannot adequately serve the needs of food processors and regulatory agencies. Hospitals typically use their own laboratories for identifying bacterial pathogens, whereby a swab of urine, sputum, or blood sample is sent to a laboratory. Tests are then performed to determine if the pathogens are present. Conventional testing frequently requires 24 hours, and with laboratory backlogs, results may frequently take up to days.

The lack of speed and uncertainty common to existing medical and microbiological pathogen detection and identification procedures have led to ever-increasing rates of food-borne and water-borne contamination outbreaks and the over-prescription and abuse of antibiotics, leading to significantly increased occurrences of multiply-drug resistant bacteria. These testing procedures also unfortunately suffer from personnel safety issues and a high cost.

About 3.1 million blood cultures were ordered annually in US Emergency Departments from 2001-2004, in 2.8% of all visits, excluding non-emergency testing. Almost half of these patients were discharged home, and many did not appear to have indications for blood cultures. Yet, blood cultures have an integral role in the evaluation of febrile patients. Approximately 200,000 cases of bacteremia occur each year, with an associated mortality rate of 40%-50%. As a result, clinicians have been encouraged to obtain blood samples for culture from febrile patients. Conventionally, the results of the majority of these blood cultures were predicted on the basis of case histories, rarely identifying unexpected pathogens, putting a serious strain on hospital resources for very little return. In fact blood cultures generally produce useful information for only a small subset of admitted patients but large health care oversight organizations consider them a marker of “quality” of care. The cost to patients and institutions is large, but could be reduced if a proper rapid yet inexpensive screening technology was implemented.

Current technology for the most part requires bacterial samples to be incubated and grown on or in specific nutrition media for a considerable period of time (usually days) to achieve a high enough concentration (or titer) before any form of identification of the bacteria can be attempted. This culture-and-count method, combined with phenotypic identification based on the Gram-stain and growth on selective media, sometimes augmented with multiwell antigenic identification, is currently the gold-standard for bacterial identification.

In this context of increasing rates of pathogen infection, the emergence of new multiply-drug resistant microorganisms, troubling increases in the rates of hospital-acquired infections, and increasing occurrences of foodborne contamination, the demands on clinical and public health microbiology laboratories will only continue to increase in the 21^{st} Century. Laboratories will be challenged to not only diagnose these ever-increasing incidents of bacterial infection, but to also detect/identify new or emerging pathogens which may arise by mutation of existing organisms or introduced by bioterrorism. Although there are several reliable and accurate techniques for identifying pathogenic bacteria, as well as several new and emerging candidate techniques, to date the microbiological expertise and cost required to perform bacterial identifications preclude their common use as a rapid screening mechanism to prevent human infection. A rapid, inexpensive bacterial identification technology for high-throughput screening of specimens that does not rely on a priori knowledge of bacterial genetic composition (DNA-based methods) or antigenic variation (serological or antigenic-based methods) is therefore desperately needed.

Recently there have been attempts to use laser-induced breakdown spectroscopy (LIBS) technology for the analysis of biological samples or cultures, as for example is described in the inventor's earlier publication J. Diedrich, S. J. Rehse and S. Palchaudhuri, “Pathogenic Escherichia coli Strain Discrimination Using Laser-Induced Breakdown Spectroscopy,” J. Appl. Phys. 102, 014702 (2007). LIBS technology performs a rapid elemental assay of a specimen utilizing a laser-created high-temperature spark. U.S. Pat. No. 5,583,634 A, Andre et al. Assignee: Commissariat a l′Energie Atomique. 1996, entitled “Process for elementary analysis by optical emission spectrometry on plasma produce by a laser in the presence of argon”, describes a LIBS method for creating a spark on a sample immersed in an argon gas stream for the purpose of determining the composition of a sample. Andre does not specify any type of target, nor the performance of LIBS on bacteria. United States Patent Publication No. US2009/0273782A1, to Yoo et al, entitled “Laser ablation apparatus and method”, describes an apparatus and method for performing laser ablation spectroscopy. Yoo, however, does not relate any particular type of sample, nor discloses performing LIBS on bacteria.

U.S. Pat. No. 7,999,928 B2, to Beckstead et al. entitled “Method and system for combined Raman and LIBS detection”, describes a method and system for identifying a sample using its LIBS spectrum and/or Raman spectrum. Beckstead does not relate specifically to any particular type of sample, nor perform LIBS on bacteria.

United States Patent Publication No. US2011/0246145A1, to Multari et al. 2011, entitled “Methods for forming recognition algorithms for laser-induced breakdown spectroscopy”, teaches a very specific computer chemometric algorithm (a computer program) to accurately differentiate LIBS spectra. Multari broadly suggests analyzing spectra from LIBS performed on solid, liquids, aerosols, soil, bacteria, explosives, and metals; and in particular teaches a method of computerized chemometric analysis, using a specific way of sorting the data beforehand to identify relevant target and test spectra. Multari however is of little assistance in teaching the use of LIBS on bacteria.

United States Patent Publication No. US2011/0171636A1, to Melikechi et al., entitled “Mono- and multi-element coded LIBS assays and methods”, teaches a method for “tagging” objects with unique elemental-coded markers or tags prior to LIBS analysis to enable quick identification of the tagged target. Melikechi describes the analysis of biological and chemical molecules, and references third party papers suggesting the use of LIBS on bacteria. Melikechi however does not expressly describe the performance of LIBS on bacteria or its possible use in the identification and classification.

Matrix-assisted laser desorption/ionization-time of flight mass spectrometry (MALDI-TOF-MS) currently may provide a similar physico-chemical technique for fast and reliable microorganism identification. The applicant has recognized LIBS is considerably easier to implement (no vacuum system, no mass spectrometry), less expensive, and utilizes an apparatus that is smaller by about an order of magnitude as contrasted with the MALDI-TOF-MS apparatus. The LIBS device of the present invention will be more cost effective by virtue of being highly convenient (even portable), useable by non-experts, and providing easy to interpret results.

LIBS has been demonstrated to be useful when used in laboratory settings to discriminate between microorganisms: bacteria, mold, pollens, fungal spores, and bacterial spores. Heretofore, such discrimination has been based on the atomic emission strength of inorganic elements, as well as carbon. To date, an issue with LIBS includes that bacteria are so small that the laser, along with the bacteria, breaks down the material that is supporting the bacteria. As a result, the produced spectrum shows both the bacteria and the support material, skewing the results with the added material reading and compromising the analysis.

### SUMMARY OF THE INVENTION

One non-limiting object is therefore to provide a commercial LIBS instrument for use in diagnostic microbiology laboratories in the diagnosis of clinical pathogens, and more preferably bacteria.

The present invention provides a new rapid biosensing/pathogen detection system which may broadly encompass several scientific/health food communities. More preferably, the present invention provides a method for the use of laser-induced breakdown spectroscopy to identify pathogens, and preferably bacteria in such communities, wherein the method includes one or more steps of:
1. Collecting bacterial colonies from one or more of:
   (i) a plate of cultured bacteria;
   (ii) a clinical human specimen, including but not limited to: blood, sputum, urine, stool, cerebral spinal fluid;
   (iii) a water sample;
   (iv) the surface of any food time or consumable foodstuff either via washing, swabbing, rinsing; and/or
   (v) any non-sterile surface via swabbing, rinsing, or washing; and then
2. Preparing the collected bacteria via suspension in any other liquid media, including but not limited to water, phosphate buffered saline, or any other growth medium, either with or without a washing step;
3. Filtering the liquid sample through a suitable microbiological filter medium; and/or deposition of the liquid sample on a suitable microbiological filter medium; and/or any other form of filtration step to separate the bacteria from the liquid, or
4. Effecting centrifugation of the liquid sample and collection of the bacterial pellet so formed, for the purpose of forming a bacterial pellet; or
5. Effecting separation of bacterial cells from any liquid sample including but not limited to: blood, sputum, urine, stool, cerebral spinal fluid, drinking water, or liquid used to wash a sample by use of a dielectrophoresis channel apparatus; or
6. Effecting separation of bacterial cells from any liquid sample including but not limited to: blood, sputum, urine, stool, cerebral spinal fluid, drinking water, or liquid used to wash a sample by use of a hydrodynamic cell-sorting microfluidic channel apparatus; or
7. Effecting separation of bacterial cells from any liquid sample including but not limited to: blood, sputum, urine, stool, cerebral spinal fluid, drinking water, or liquid used to wash a sample by means of a cell-sorting fluidic channel utilizing biomolecular bonding with bacterial phages or antibodies; and then
8. After obtaining the bacteria, depositing the bacteria so obtained on a sample holder such as a nutrient free agar substrate, agar microscope slide, microbiological filter, a disposable or permanent sampling holder, or alternately, leaving the swabbed bacteria directly on the swab or wipe, or within or on a microchannel device with or without dielectrophoresis separation; and then
9. Determining the atomic composition of the bacterial specimen so obtained via laser-induced breakdown spectroscopy by an analysis of an obtained atomic emission spectral fingerprint; and then
10. Comparing the atomic emission spectral fingerprint so obtained, against a pre-compiled library of spectral fingerprints from previously identified organisms, for the purpose of matching the unknown spectral fingerprint, and identifying the obtained spectral fingerprint using a chemometric algorithm. The chemometric algorithm may optionally include one or more discriminant function analysis, partial least squares-discriminant analysis, principal component analysis, neural network algorithms; and
11. Conveying the results of the computerized matching classification to a user/operator which may or may not include the statistical uncertainty of the identification or the confidence of the identification.

It is envisioned that the foregoing testing methodology, whilst preferably used in the analysis of bacterial specimens, may also be valid for a variety of pathogens, including without restriction spores, pollens, molds, mildews, and viruses.

In another preferred method, LIBS is effected on bacteria directly obtained from a centrifuged urine sample for the purpose of identifying urinary tract infections.

In one possible embodiment, the invention provides a pathogen detection system including a specimen support for supporting a test specimen sample to be analyzed, a coherent light source, an optical detector and an analyzer electronically coupled to the optical detection. The coherent light source is operable to direct a coherent light beam at the specimen support to break down and at least partially atomize said test specimen sample. The optical detector is positioned to detect a spectral signature of electromagnetic radiation emitted by the partial atomization of the test specimen sample and electrically communicate the detected spectral signature to the analyzer. The analyzer compares the detected spectral signature to one or more predetermined spectral signatures for one or more pathogens.

In another embodiment, the invention resides in a method that exploits laser-induced breakdown spectroscopy (LIBS) to provide a rapid identification of pathogens, and more preferably bacteria, based on their unique atomic composition. One primary advantage of the present system is speed. Identification may preferably be achieved in less than a few minutes, to as little as several seconds, once samples are prepared. In a preferred automated mode, the time taken for the laser to fire, for optical emission data to be collected, and for a computer using a custom algorithm to match the detected new “unknown” spectrum against the pre-compiled library of “known” spectra may be from a few seconds to a few minutes. Ultimately it is envisioned that the whole testing process, from obtaining a clinical specimen to obtaining the result, could take less than sixty, and preferably in under 5 minutes. In contrast, methods currently used in clinical settings to identify unknown pathogens typically take 24-72 hours, and may be too time-consuming to direct immediate patient treatment.

In another embodiment, the invention provides for a system operable to use LIBS to determine the atomic composition of the bacteria in real-time, and which bridges the current identification schemes of molecular DNA-based technologies and traditional, but unavoidable, microbiological/immunological techniques. More preferably an atomic-based biosensing technology is provided which is operable to output substantially immediate diagnostic information for multiple pathogens. It is to be appreciated that providing this information in the hands of medical professionals may help reduce the crisis in antibiotic resistance and patient suffering. The present invention may also act as a preliminary autonomous screening technology in the food industry, to reduce occurrences of food poisoning, and/or ensure the safety of water for drinking and recreation at beaches, lakes, and oceans.

More preferably, for increased accuracy using LIBS analysis, the bacteria to be analyzed are concentrated to a concentration of over 50% by cell-count titer, preferably over 70% by cell-count titer, and more preferably to over 80% by cell-count titer by the use of a microfluidic device; a microchannel device; biomolecular bonding bacterial phages or antibodies; filtration; and/or centrifugation. The material used to hold the sample during LIBS testing is further preferably selected so as not to contain and/or produce an atomic signature within or close to the expected atomic element signatures of the specimen.

In a preferred method, to ensure that the machine is calibrated to accommodate the material for supporting the sample, and to provide the software system of the apparatus a graphic comparison against which to identify the bacteria being tested, a full library of test spectrums is created for the bacteria and support material combination for which the apparatus will be used. The inventor has appreciated that various families of bacteria exhibit similarities in their element signatures in the LIBS process. Thus, even new unknown forms of bacteria may be associated to existing known bacteria through the signature similarities.

In another embodiment, the invention provides a method of using the LIBS system to provide evaluations of various treatment protocols for any given pathogen or bacteria culture. In a preferred embodiment, a sample would be prepared as above, to concentrate the bacteria. Following concentration, the sample is separated into a number of equal sized test samples or fractions. A first test sample is tested to identify the bacteria, with the intensity of the reading providing an initial indication of bacteria concentration. More preferably, the first test sample is placed on a non-growth medium to provide a base line for bacteria concentration. Subsequent other control test samples are placed on a growth medium; and remaining test samples are placed onto mediums that contain different antibiotics. The test samples are then placed into a suitable growth environment for a selected period of time, to allow cell replication (preferably cell doubling or more). After cell growth, the individual test samples are then LIBS tested. The applicant has appreciated that a resulting intensity reading equal to or less than the base-line sample would show an effective response to drug or antibiotic treatment. Similarly, an intensity reading equal to or slightly less than the control growth medium sample may thus indicate no significant effect by the drug antibiotic.

It is envisioned that the present invention provides for various preferred analytical uses. For example, Streptococci A and B as well as “super bugs” are serious health threats to children and expectant mothers. Utilizing the traditional method of testing, which takes between 24 to 48 hours, may result in the advancing of the infection within the patient. A LIBS system in accordance with the present invention is generally simpler to operate and maintain due to the lack of a complicated vacuum system and magnets (required in a mass-spectrometer). In addition, the cost of testing samples or isolates is typically lower, as the time per isolate will be significantly reduced, and the experience and qualification of the operator(s) will not need to be as high due to the greater simplicity of the LIBS instrument. It is further envisioned that the LIBS system will require a much smaller laboratory footprint, and less electrical infrastructure.

The LIB S-based system of the present invention may provide clinicians with a substantially real-time diagnostic that can be used at the point of care for immediate patient diagnosis and detection of NIAID Category A, B, or C priority pathogens. LIBS-based identification does not require the culturing of any samples, the knowledge of DNA sequences, PCR primers, 16s rRNA probes, or fluorescent antibodies. The benefits of being able to determine if a pathogen is present in a clinical specimen and to identify that pathogen rapidly, will ultimately help clinicians to make a better informed infection diagnosis (even in pre-symptomatic individuals). This in turn will facilitate the proper antibiotic treatment to be initiated, and may eliminate the over-use of broad spectrum antibiotics.

Due to its speed and flexibility, the present system has applications across a number of fields including medical microbiology, environmental epidemiology, tumor/cell biology, and public health.

By speeding diagnosis, the LIBS system of the present application may advantageously minimize the suffering of patients and reduce hospital stay times by speeding diagnoses and treatment of infectious disease. Clinical usage may advantageously allow for rapid identification of causative organisms (combined with medical case histories, symptoms, etc.) allowing practitioners to decide if the patients need treatment. The output of the LIBS system may be statistically defined by a computerized algorithm and presented in a way immediately useful to physicians, nurses, and other health care professionals. Simultaneously, samples could be sent for traditional testing, and 24 hours later the existing technologies may confirm the diagnosis.

The emergence of antibiotic resistant bacteria is a serious problem in clinical medicine. In an alternate use, the present system may be used to identify the pathogens in clinical samples at “time zero” and rapidly confirm, preferably within as little as two hours of obtaining a patient specimen, if the bacteria have become resistant to the antibiotics of choice. For example, Staphylococcus aureus is a very common pathogen and in an increasing number of cases, the resistant strain known as “MRSA” has become a serious problem. If it was known before treatment that the strain was resistant, a more appropriate patient treatment plan would be put in place, saving millions both treatment costs and time, which equates to a reduction in mortality and suffering.

In a more advanced protocol, antibiotic resistance utilizing the present LIBS-based system may form the basis of a diagnostic test, administered immediately at the point-of-care, which will not only detect and identify pathogens, but also provide the clinician with guidance about the proper prescription of the drugs of choice.

In an alternate use, testing and treatment of urinary tract infections (UTI's) is achieved using the LIBS-based system of testing on bacterial specimens obtained from urine samples. The present invention may thus provide a rapid cost-effective method of UTI diagnosis as an alternative to culture to combat this infection; which is also the most common source of infection in children under five. In particular, LIBS-based diagnostic technology advantageously allows for a rapid, effective identification of bacteria in blood, urine, sputum, and spinal fluid and thus could constitute a time-saving solution to all of the above-mentioned area of clinical diagnosis (and many more) by forming a “platform-technology” on which a variety of tests may be based, and administered directly at the “point-of-care” after a one-time hospital investment in instrumentation.

By way of non-limiting example, the present invention provides at least the following aspects.

1. In a first aspect, a pathogen detection system comprising, a specimen support for supporting a test specimen sample to be analyzed, a coherent light source operable to direct a coherent light beam at said specimen support to at least partially atomize said test specimen sample, an optical detector positioned for detecting a spectral signature of electromagnetic radiation emitted, reflected, or absorbed by the at least partial atomization of the test specimen sample, an analyzer for comparing the detected spectral signature to at least one predetermined spectral signature for one or more pathogens.
2. In a second aspect, a method of determining pathogen drug or antibiotic resistance using a laser-induced breakdown spectroscopy system comprising a specimen support for supporting test specimen samples to be analyzed thereon, a coherent light source operable to direct a coherent light beam at said specimen support to at least partially atomize said test specimen samples, an optical detector positioned for detecting the spectral signatures of electromagnetic radiation emitted by the at least partial atomization of a selected test specimen sample, and an analyzer operable to compare the detected spectral signature to at least one stored spectral signature of another specimen sample, said method comprising the steps of providing a first test specimen sample representative of an untreated, or drug or antibody treated sample at a first period of time, with said first test specimen sample on said specimen support, actuating said coherent light source to at least partially atomize said first test specimen sample, with said optical detector, detecting a spectral signature of electromagnetic radiation emitted by the at least partial atomization of said first test specimen sample as one said stored spectral signature, providing a second test specimen sample representative of the drug or antibody treated sample at a second period of time, with said second test specimen sample on said specimen support, actuating said coherent light source to at least partially atomize said second test specimen sample, with said optical detector, detecting the spectral signature of electromagnetic radiation emitted by the at least partial atomization of said second test specimen sample, and with said analyzer, comparing an intensity of the spectral signature of the second test specimen sample with an intensity of the stored spectral signature.
3. An aspect according to any of the foregoing aspects, wherein the specimen sample is selected from the group consisting of a bacterial culture, a tissue biopsy and a body fluid specimen.
4. An aspect according to any of the foregoing aspects, wherein the pathogen comprises a bacteria.
5. An aspect according to any of the foregoing aspects, wherein said coherent light beam comprises a pulsed laser beam.
6. An aspect according to any of the foregoing aspects, wherein the test specimen sample comprises one of a plurality of test samples prepared from a sample.
7. An aspect according to any of the foregoing aspects, wherein the analyzer is further operable to compare an intensity of the detected spectral signature of the test specimen sample with an intensity of a stored spectral signature of at least one other of said test samples.
8. An aspect according to any of the foregoing aspects, wherein said coherent light beam has a focused beam diameter at said specimen support selected at less than about 250 μm, and preferably at about 100 μm.
9. An aspect according to any of the foregoing aspects, wherein said test specimen sample comprises bacteria cells concentrated to at least about 50% by cell-count titer, and preferably at least 70% by cell-count titer.
10. An aspect according to any of the foregoing aspects, wherein providing said test specimen sample on said specimen support, actuating said coherent light source to generate said coherent light beam to at least partially atomize said test specimen sample to effect the emission reflection and/or absorption of electromagnetic radiation with said optical detector, collecting and storing a detected spectral signature of said electromagnetic radiation, and comparing the detected spectral signature of said test specimen sample with one or more of said predetermined spectral signatures.
11. An aspect according to any of the foregoing aspects comprising a step of concentrating said pathogen content in a sample, and dividing said sample into a plurality of substantially equally sized fractions, and selecting one of said fractions as said test specimen sample.
12. An aspect accordingly to any of the foregoing aspects, wherein said pathogen comprises bacteria, and further comprising concentrating said bacteria to a concentration of at least about 70% by cell-count titer, and preferably at least about 80% by cell-count titer, prior to providing said pathogen in said test specimen sample.
13. An aspect according to any of the foregoing aspects, wherein said test specimen sample comprises a culture media selected from the group consisting of TSA, MAC and deoxycholate-spiked agar.
14. An aspect according to any of the foregoing aspects, wherein said coherent light beam is activated to produce a pulsed beam having a beam diameter at said test specimen sample of between 50 μm and 150 μm, and preferably between about 75 and 125 μm.
15. An aspect according to any of the foregoing aspects further comprising outputting an increase in the intensity of spectral signature of the second test specimen sample as an indication of an increase in pathogen resistance to said drug or antibody.
16. An aspect according to any of the foregoing aspects, wherein the bacteria is selected from the group consisting of Escherichia, Sthphylococcus, S. viridans, S. epidermidis, E. cloacae, M. smegmatis Bacillus anthracis and C. difficile.
17. An aspect according to any of the foregoing aspects, comprising an earlier step of concentrating said pathogen content in a sample, and preferably concentrated to at least about 70% by cell-count titer, and dividing said sample into a plurality of substantially equally sized fractions, and selecting individual ones of said fractions as said first and second test specimen sample.
18. An aspect according to any of the foregoing aspects, wherein said test specimen sample comprises a sample support selected from a liquid nutrient medium and a lysogeny broth.
19. An aspect according to any of the foregoing aspects, wherein said specimen support comprises a purified agar support having between about 1 and 2% by wt solid culture media.
20. An aspect according to any of the foregoing aspects, wherein said analyzer compares the detected spectral signature of the test specimen sample with at least one of a detected or predetermined spectral signature of the specimen support.

### BRIEF DESCRIPTION OF THE DRAWINGS

Reference may be had to the following detailed description taken together with the accompanying drawings, in which:

FIG. 1 shows schematically a laser-induced breakdown spectroscopy system for detecting bacteria in a test specimen sample in accordance with the preferred embodiment of the invention;

FIG. 2a shows schematically a sample output spectral signature of the electromagnetic radiation emitted upon atomization of a test specimen containing sample bacteria cells;

FIG. 2b shows schematically the output spectral signature of the electromagnetic radiation emitted upon atomization of the substrate support used in the test sample of FIG. 2a;

FIGS. 3a and 3b show schematically the output analyzed spectral data of LIBS atomized Escherichia coli and Pseudomonas aeruginosa bacteria test samples prepared using TSA, MAC and Deoxycholate-spiked agar;

FIG. 4 shows schematically the output analyzed spectral data of LIBS atomized Streptococcus viridans and Escherichia coli bacteria test samples over separate time intervals;

FIGS. 5a and 5d show schematically the output analyzed spectral data of LIBS atomized Streptococcus viridans and Escherichia coli bacteria test samples exposed to bactericidal UV light and autoclaved prior to mounting on agar plates;

FIG. 6 shows graphically the LIBS spectral intensity output relationship to bacterial cell concentration;

FIGS. 7a to 7c show schematically the LIBS output analyzed spectral data of different genera of bacteria;

FIGS. 8a to 8c show schematically the output analyzed spectral data for LIBS atomized test samples containing mixtures of M. snieginatis, E. coli and E. cloacae bacteria; and

FIGS. 9a and 9b show schematically the output analyzed spectral data for LIBS atomized test bacteria samples containing Staph epidermidis, E. coli, and S. viridans.

### DETAILED DESCRIPTION OF THE PREFERRED EMBODIMENTS

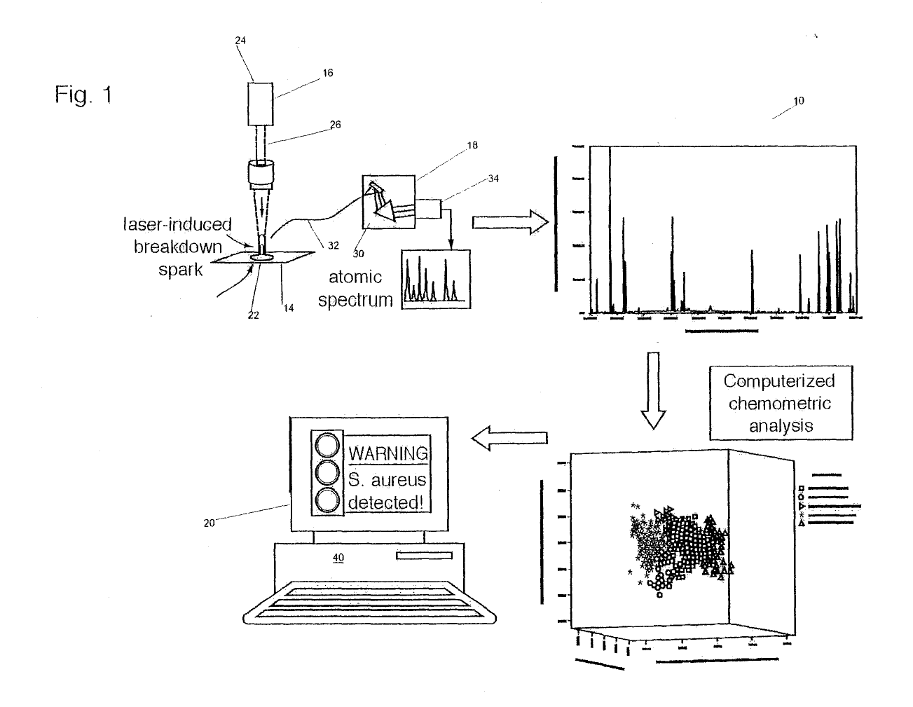

As shown best in FIG. 1, the present invention provides a laser-induced breakdown spectroscopy (LIBS) system 10 which utilizes laser-based spectroscopic assay/diagnostic technology to enable the rapid, and preferably under 1 second, and accurate (sensitivity and specificity in excess of 95%) identification of various types of bacteria and/or environmental and clinically relevant microorganisms. As will be described LIBS system 10 is operable to effect in-situ measurements of atomic or elemental composition of bacterial cells with a bacterial culture, tissue biopsy or body or other fluid specimen.

FIG. 1 shows the LIBS system 10 as including a test sample support 14, a laser assembly 16, and an optical collector assembly 18 which is directly or indirectly electronically connected with a CPU analyzer 20. The test sample support 14 is configured to support a selected sample specimen 22 which contains a bacteria culture, and preferably a bacteria culture concentrated to at least about 70% Titer. Preferably, the sample support 14 is selected so that if partially or wholly atomized, the sample support 14 neither contains nor produces an atomic signature which is within or close to the anticipated atomic element signature of the bacteria cultured to be assayed. The applicant has appreciated that most preferred test sample supports 14 therefore include, nutrient free BD Bacto™ agar substrate (Becton, Dickenson and Company), as well as other gelatinous media consisting of 97% by wt or more, and preferably 99% water; and upto 3% and preferably about 1% by wt Dehydrated Culture Media. More preferably the support medium used as the sample support 14 is purified to reduce to a minimum extraneous matter, pigmented portions and salts.

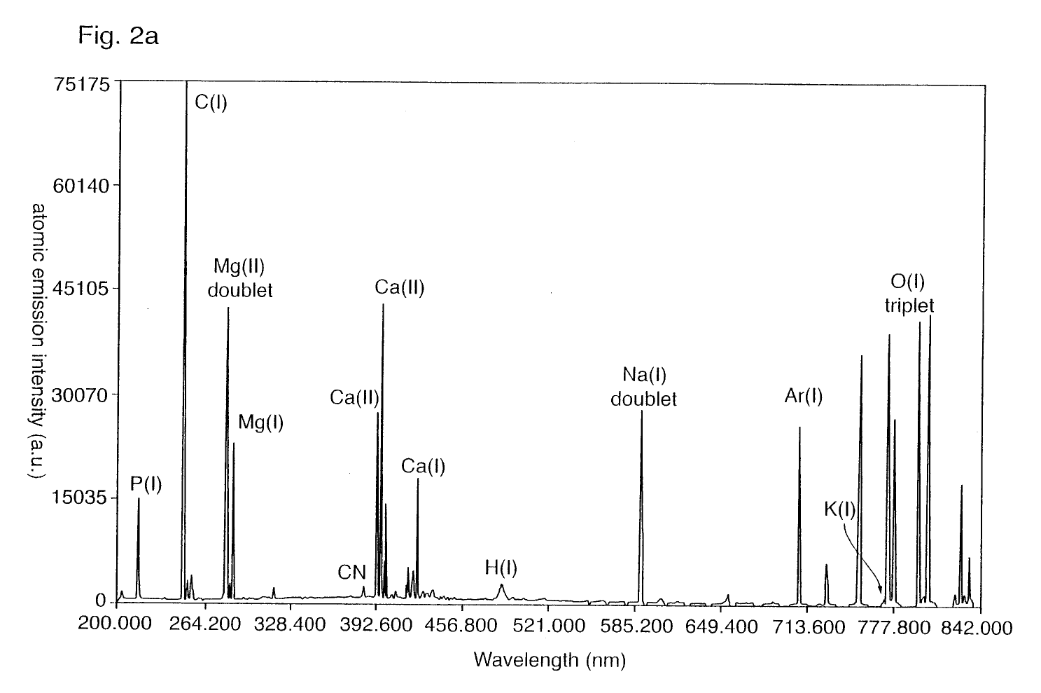

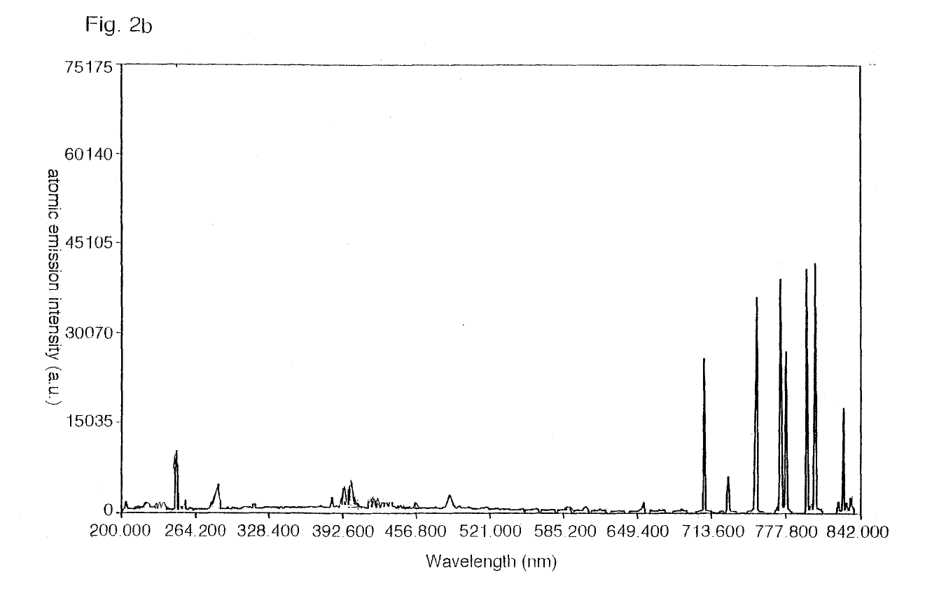

The laser assembly 16 is shown in FIG. 1 as including a coherent light source 24 which is operable to selectively emit a pulsed laser beam 26, and a microscope objective lens 28. The lens 28 is positioned to focus the pulsed laser beam 26 as a 50 to 150 micron diameter high energy beam at the point of the test sample support 14. The laser assembly 16 is operable to provide a focus high energy pulsed laser beam 26 at the surface of the test sample support 14 to effect the laser atomization of the test sample 22 containing the bacterial cells to be analyzed thereon. Upon laser atomization of the test sample 22, a laser-induced breakdown spark is achieved on sample vapourization which produces a spectral signature, which for example is shown in FIG. 2a.

More preferably, the sample support 14 is chosen so that when atomized, the sample support 14 itself yields a predetermined and/or known. An exemplary spectral signature for a nutrient free bacto agar sample support 14 is shown in FIG. 2b, and may be pre-stored in the CPU analyzer memory. The spectral signature of the sample support 14 may thus be rapidly and easily filtered and substrated from the active detected spectral signature of the bacteria shown in FIG. 2a by the analyzer 20.

The optical collector assembly 18 is operable to detect and collect any electromagnetic radiation which is produced in the breakdown spark upon the atomization of the test sample 22 by the emitted laser beam 26. In a preferred construction, the optical collector assembly 18 includes a spectrometer and preferably an echelle grating spectrometer 30 which is optically connected to each of a fiber optic cable 32 and CCD camera 34. The fiber optic cable 32 extends from a position immediately adjacent to the area of the test sample support 14 which is to be irradiated by the pulsed laser 26 for the accurate transmission of any produced electromagnetic energy flash to the spectrometer 30, where it is then output the CCD camera 34 and CPU analyzer 20. The CPU analyzer 20 may be electrically coupled directly to the CCD camera 34. In an alternate configuration, the CPU analyzer 20 may be provided at a remote location and configured to electrically receive and/or transmit data from and to the optical collector assembly wirelessly and/or via a computer network.

The CPU analyzer 20 is operable to provide a visual output of the spectral fingerprint of the emitted electromagnetic energy produced upon the atomization of a test sample 22 on the sample support 14 by the pulsed laser beam 26. More preferably, the CPU analyzer 20 is provided with memory 40 operable to store and/or compare a detected spectral signature for a given test sample 22 to one or more spectral signatures of bacteria which have been previously determined, and which are stored in the CPU memory 40 as part of a spectral signature library. The CPU analyzer 20 is preferably operable to store the spectral signatures for multiple test samples, as for example to permit their comparison as a measurement of bacterial or other pathogen activity over time.

The LIBS system 10 of FIG. 1 may be used in the analysis of a number of different types of bacterial-containing test specimens 22. Such test specimens 22 may be presented in a variety of different forms and, for example, may include without restriction bacterial cultures, tissue biopsies, and body fluid specimens such as blood, urine, CSF or the like. In use of the system 10, bacteria cells in test sample specimens 22 are provided on the sample support 14.

In exemplary testing, the bacteria cells were transferred to the surface of a nutrient free 1.4% bacto agar plates as sample supports 14. Individual sample supports 14 were prepared for cell seeding, whereby:
1. A suitable (i.e. Bacto™) agar powder was mixed with 100 to 150 ml of distilled water.
2. The solution was boiled and stirred at a speed of about 120 rpm for 10 minutes, on a hot plate, at a temperature of about 300° C.
3. The boiled solution was then poured into individual small petri dishes.
4. The top surface of each petri dish was scraped to ensure a level and uniform agar surface for accurate LIBS results.
5. The petri dishes were stored in a refrigerator to cool down for at least 30 minutes. Typically, individual agar plates were prepared 24 hours before bacteria deposition for LIBS testing.
6. Ten microliters of a high-density bacterial suspension (pellet) were micropipetted onto the bacto-agar surface to distribute the bacteria generally evenly over the surface of the agar plate. After approximately thirty minutes, the liquid was shown to be absorbed by the agar, leaving a transparent thin film or “bed” of bacteria approximately 0.5 cm² in area.
7. The petri dish and bacteria was thereafter optionally incubated for the desired time period to allow for desired culture maturation, and provide the desired test specimen 22. After a selected period of incubation, each petri dish was moved to the laser assembly 16 as a test specimen 22.

Following positioning of the test specimen 22, the laser assembly 16 is operated to actuate the light source 26 to atomize the test specimen 22, using the produced high energy pulsed laser beam 26.

In one preferred mode, during laser-induced breakdowns spectroscopy (LIBS), a short pulse of laser light 26 is focused to a 100 μm diameter spot on a bacteria-containing test sample 22. The absorbed laser energy is converted into heat, resulting in the atomization and ablation of the bacteria cells in the 100 μm diameter focused area of the laser beam 26. The output laser energy is preferably selected to heat the ablated atoms until it creates a very high-temperature (50,000 K) plasma cloud of glowing atoms, ions, and electrons similar to a fluorescent light tube or spark. Most preferably, the coherent light source 24 operates to output a laser beam 26 with sufficient energy such that bacteria cell hit by the laser beam 26 are completely reduced to their constituent atomic components, contained within this plasma.

Upon atomization of the test sample specimen 22, an emitted light (or other electromagnetic energy) is transmitted by the fiber optic cable 32 to the spectrometer 30 and CCD camera 34. The fiber optic cable 32 is positioned adjacent the sample support 14 so as to be near the glowing plasma, to catch and transmit light to the spectrometer 30 for analysis. The analysis of the transmitted light energy yields identifiable emission lines from the elements present in the target bacteria cells. Further, the identification and intensity of all of the elemental lines within the emission spectrum may advantageously be used to rapidly provide a spectral atomic emission “fingerprint” which is unique to the bacteria. Furthermore, it has been recognized that the intensity of a line in the emission spectra is proportional to the concentration of elements in the bacterium, and is also proportional to the absolute number of bacteria that are ablated. The technique is shown schematically in FIG. 1.

The atomic composition of the test sample is thus identified by peaks in atomic emission intensity in the atomic emission spectrum. The CPU analyzer 20 is then used to analyze the detected emission spectrum and compare the specimens specific spectral characteristics of known bacteria stored in the CPU analyzer memory 40. The ratios of the intensity of the peaks thus may be used to form a spectral fingerprint unique to the test sample specimen 22, and which may permit the quantification of trace biomarkers which may be indicative of a unique pathology, disease state or condition.

With the LIBS system 10 of FIG. 1, the LIBS-based assay does not require known/conserved DNA sequences, PCR primers, nucleic acid amplification, 16s rRNA probes, fluorescently labeled antibodies, or a priori knowledge of bioinformatics. It has been appreciated that advantages over competing technologies include a substantially safer protocol, accuracy, minimal time and cost in specimen preparation, few or no consumables/perishables, relative insensitivity to specimen contamination, and a rapid diagnosis. Its ease of use, high-throughput, speed of sampling, ruggedness, and the fact that the LIBS system 10 can be made portable suggests it may advantageously be employed “point-of-care” rapid pathogen diagnostic technology for clinical laboratories, first responders, military clinics/hospitals, environmental monitors, and food-hygiene compliance laboratories.

In a most preferred mode, the present invention includes process steps to prepare bacterial test specimens for LIBS testing, and which allows the LIBS system 10 to be used for more accurate pathogen identification. In particular, as it is recognized that low-percentage inorganic elements play an important role in bacterial bioactivity.

FIG. 2a shows a typical LIBS spectrum from an E. coli cell mounted on a nutrient free bacto agar substrate, with element/composition being as follows:

TABLE 1

| Element | % of fixed salt fraction |
|---|---|
| Sodium | 2.6 |
| Potassium | 12.9 |
| Calcium | 9.1 |
| Magnesium | 5.9 |
| Phosphorus | 45.8 |
| Sulfur | 1.8 |
| Iron | 3.4 |

| Wavelength | Line |
|---|---|
| (nm) | Identification |
| 213.618 | P I |
| 214.914 | P I |
| 253.560 | P I |
| 255.326 | P I |
| 247.856 | C I |
| 279.553 | Mg II |
| 280.271 | Mg II |
| 285.213 | Mg I |
| 393.361 | Ca II |
| 396.837 | Ca II |
| 422.666 | Ca II |
| 588.995 | Na I |
| 589.593 | Na I |

As shown in FIGS. 2a and 2b the scales are about the same. In the substrate support 14 (FIG. 2b), there are very few to no emission lines of importance. The larger spectral lines common to both spectra in FIGS. 2a and 2b (the large peaks at the right) are non-specific lines of oxygen and argon, and preferably are not used in the discrimination of the bacteria. Rather is the large peaks at the left (present in FIG. 2a and absent in FIG. 2b) that allow the bacterial identification.

The elements responsible for emission lines are noted, along with their ionization states (I or II, I=neutral atom; II=singly-ionized atom) in the table above, showing thirteen atomic emission lines whose intensities are measured to construct a bacterial spectral fingerprint. The dry weight fraction of inorganic elements were determined by Luria. Currently, sulfur, iron and potassium are not observed in the LIBS spectrum, although with enhanced detector sensitivity and light collection, even more diverse elements including these will be observed.

The dry weight percentage of a variety of inorganic salts (Ca, Mg, Na, K, and Ba) in bacterial cells may thus be quantified, (see FIG. 2a). The purpose of these atoms in the bacterium is not entirely clear, however it has been recognized that inorganic elements play a role in bacterial activity. For example, the two most important elements, Ca and Mg, in the form of divalent cations, Ca²⁺ and Mg²⁺, are believed to play a role in stabilizing the outer membrane (a rugged permeability barrier) of Gram-negative bacteria by binding adjacent molecules. The outer membrane is composed mainly of an amphilic molecule which is composed of lipopolysaccharide (LPS). Without being bound by a particular theory, it is believed that Ca²⁺ and Mg²⁺ act to stabilize the entire membrane structure. The exact mechanism of cation stabilization is not yet completely clear, but it has been shown that displacement of these cation binding sites significantly alters LPS packing structure. Thus, changes in cation concentration lead to changes in membrane packing structure, which is directly related to antibiotic or antimicrobial efficacy against the bacteria, and also to bacterial serotyping. It is therefore recognized that the trace concentration of inorganic elements may play a key role in bacterial biodiversity and function and can thus be used as a reliable identifier.

The applicant has thus appreciated that by the performance of a LIBS-based analysis, it is possible to effectively measure the atomic composition of the bacteria in vitro. By pre-determining the concentrations and spectral profiles of the atoms that compose known bacteria which are closely related to serological classification via their known effect in the membrane, it therefore is possible to achieve unique bacterial identification.

As shown in FIG. 2a, the intensities of the LIBS emission peaks are not all equal. The applicant has recognized that the variations in the intensities between strains and species, or the ratios of the spectral lines in each spectrum, representing different concentrations of elements in the bacteria may advantageously form the basis of an atomic fingerprint for each bacteria. The uniqueness of this spectrum may provide a basis for an atomic composition identification or discrimination based on organism-specific non-genetic markers. The atomic fingerprint furthermore is neither phenotype-based nor antigen-antibody based, thus it is not affected by bacterial serological changes or surface ultrastructure alterations due to mutations.

Initial testing the LIBS system 10 was applied in the identification of laboratory strains of E. coli K-12, environmental strains of E. coli, pathogenic E. coli (EHEC O157:H7), Pseudomonas aeruginosa, several species of Gram-positive Staphylococci and Streptococci, Mycobacterium smegmatis, Enterobacter cloacae.

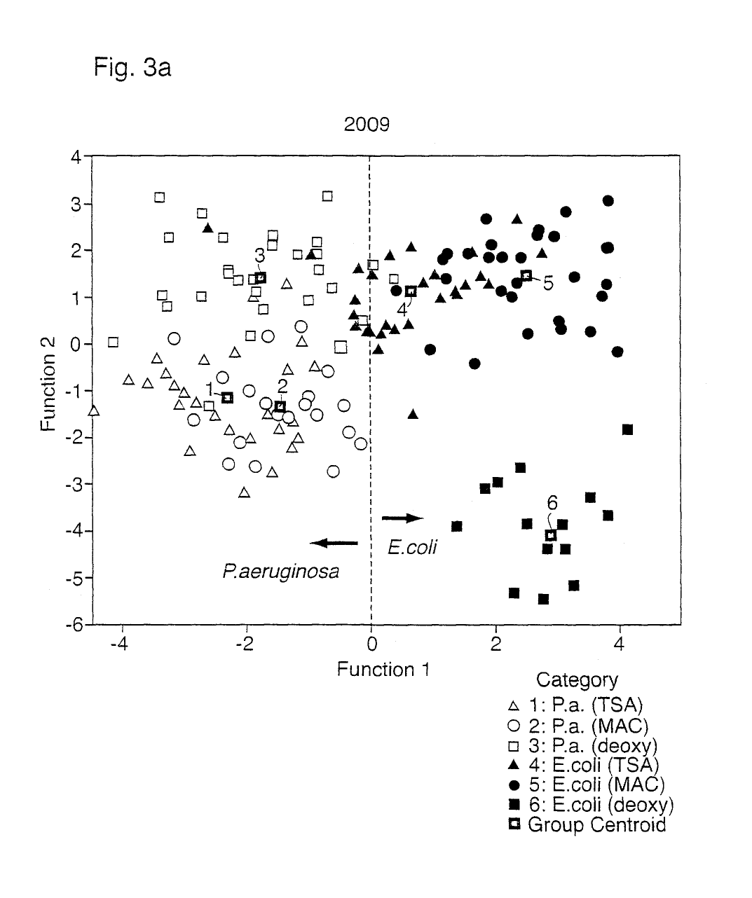

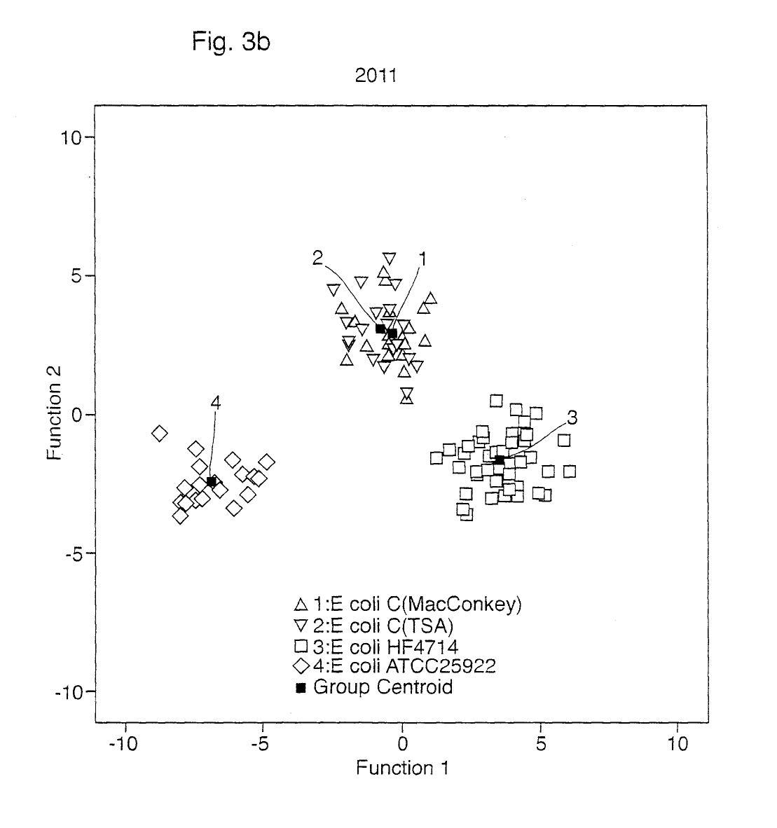

FIGS. 3a and 3b show that the LIBS-based discrimination of bacteria does not depend on culture conditions or growth medium. In the illustrated graphs, each datum represents the entire spectrum from a bacterial sample. The graphs are computerized “chemometric” analyses of spectra showing sorting/classification. Data points close to each other in the graph possess high spectral similarity. The computer may therefore be used to classify these spectra as belonging to the same organism. As shown in FIG. 3a spectra obtained from Pseudomonas aeruginosa and Escherichia coli are easy discriminated, irrespective whether the cells were cultured on a tryptic soy agar (TSA), MacConkey agar (MAC), or deoxycholate-spiked agar. On the right half of FIG. 3a three E. coli strains are differentiated. The different specimens of E. coli strain C (1 & 2) were prepared on different media—both possessed LIBS spectral fingerprints appearing identical to each other (as evidenced by their being distributed about the same point in the graph) and easily differentiated from the other strains of E. coli (because they are in a separate location on the graph.)

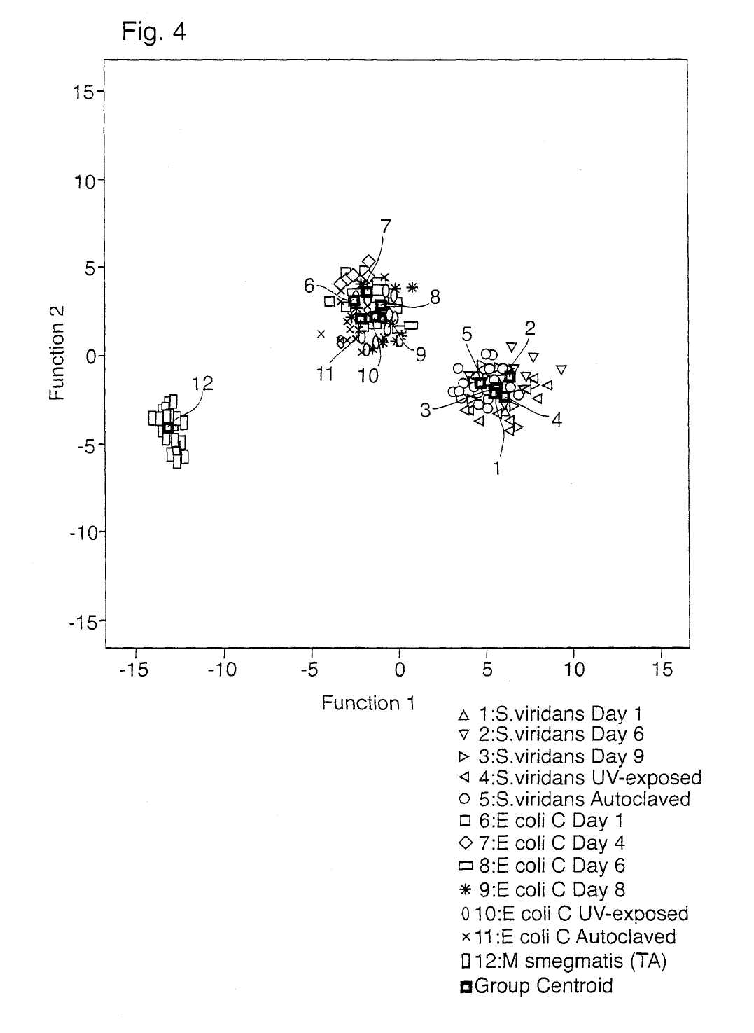

FIG. 4 shows that the LIBS-based spectral fingerprint does not change with time on abiotic surfaces. Streptococcus viridans and Escherichia coli were prepared separately and cells were mounted for up to 10 days on an abiotic agar plate prior to LIBS testing. The spectra from these cells were identical to spectra from freshly-harvested cells from a culture, and were also identical to spectra obtained from cells that were autoclaved and cells that were exposed to a bactericidal UV lamp (neither of these changing the LIBS spectrum).

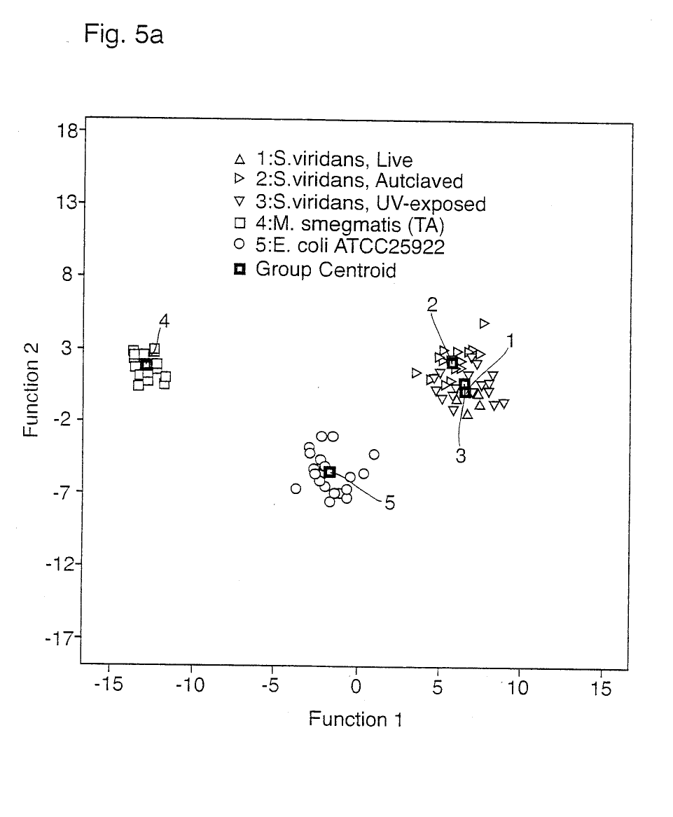

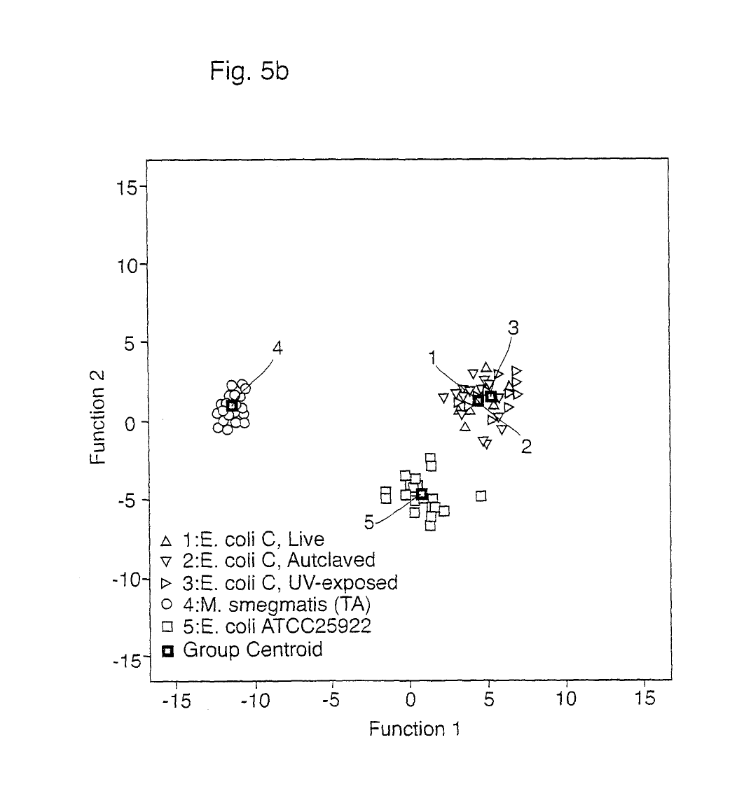

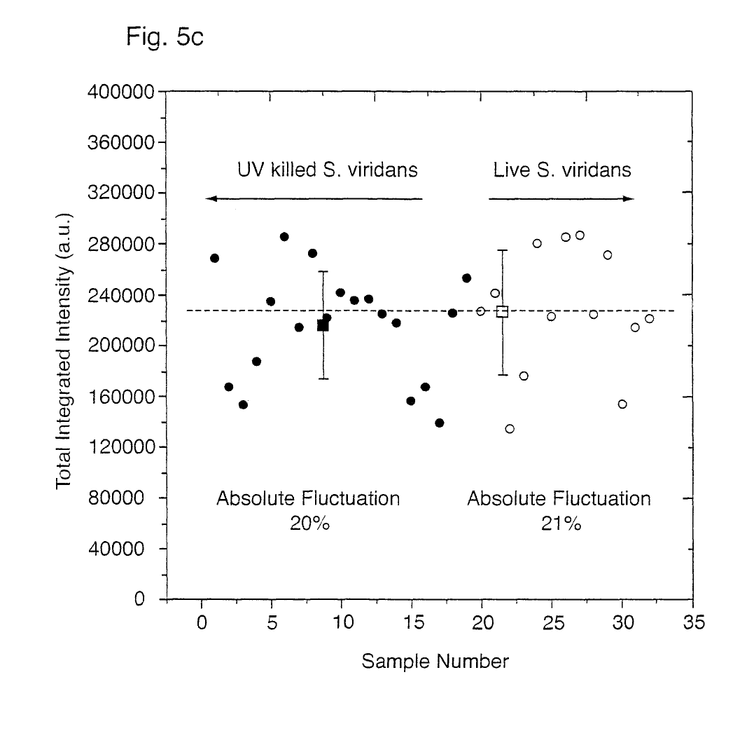

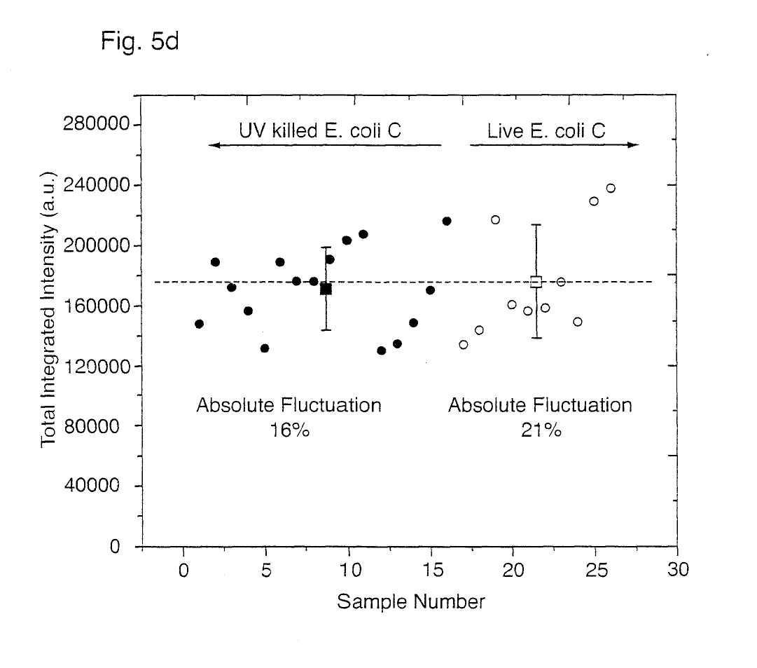

FIGS. 5a to 5d show that the LIBS-based spectral fingerprint does not change whether the cells are alive, autoclaved, or inactivated by UV light (independent of cell cycle). Streptococcus viridans (FIGS. 5a and 5c) and Escherichia coli (FIGS. 5b and 5d) were prepared separately and cells were mounted on a nutrient-free agar and exposed to bactericidal UV light, while some harvested cells were autoclaved prior to mounting on the agar plate. All cells (samples 1, 2, and 3) possessed identical spectra and were easily differentiated from other species of bacteria. The intensity of the LIBS signal did not change in killed cells (bottom) meaning the test could be performed on autoclaved specimens, rendering it far safer than alternate tests.

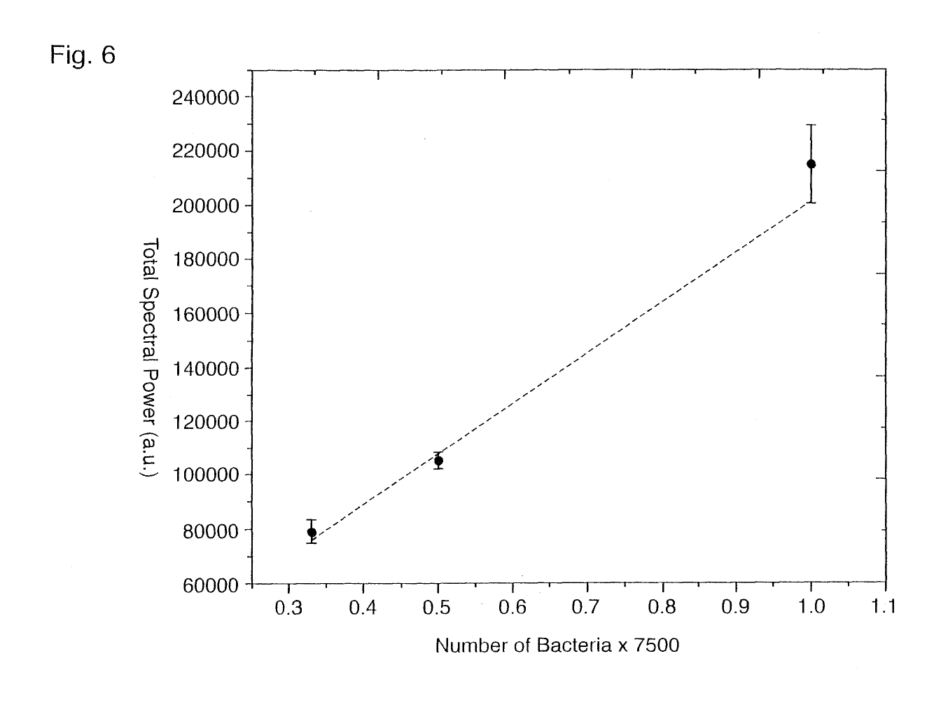

FIG. 6 illustrates graphically the intensity of the LIBS signal and being linearly dependent on bacteria cell number. By varying the titer of liquid specimens prior to mounting on the testing surface, the number of cells ablated by the LIBS laser pulse was varied. The total emission power observed in the LIBS spark was linearly dependent on the cell number. While 7500 cells yielded a high signal to noise spectrum, as few as 1500 cells provided adequate signal for accurate bacterial identification. This suggests a test for antibiotic resistance that may take less than an hour (as opposed to 24) by monitoring changes in the LIBS spectrum, as bacteria grow or do not grow on antibiotic plates during one colony doubling time.

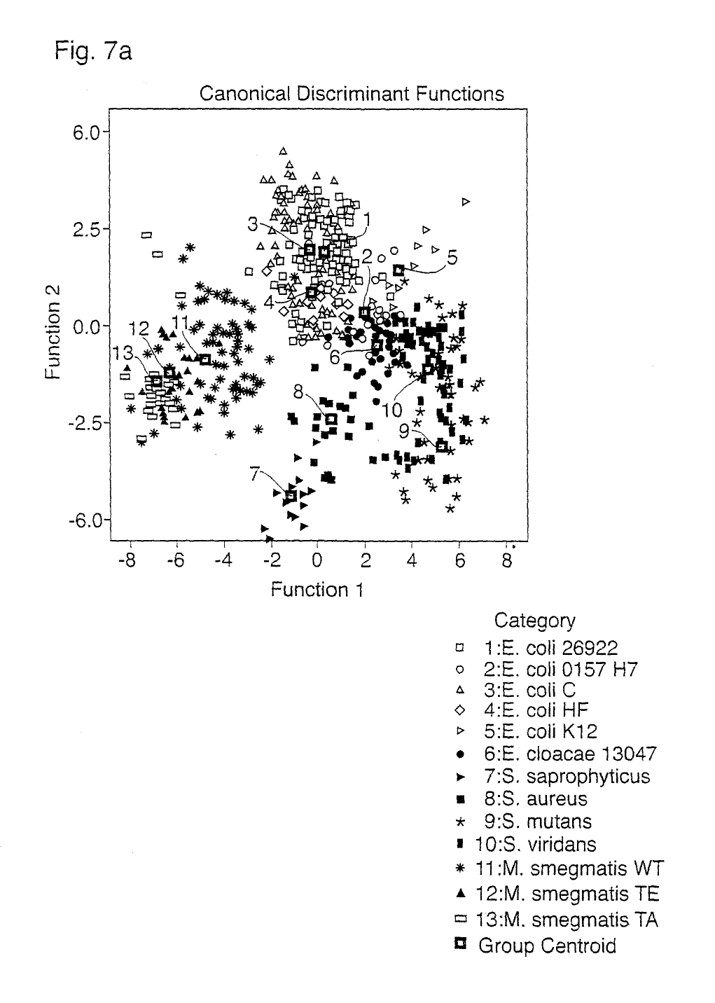

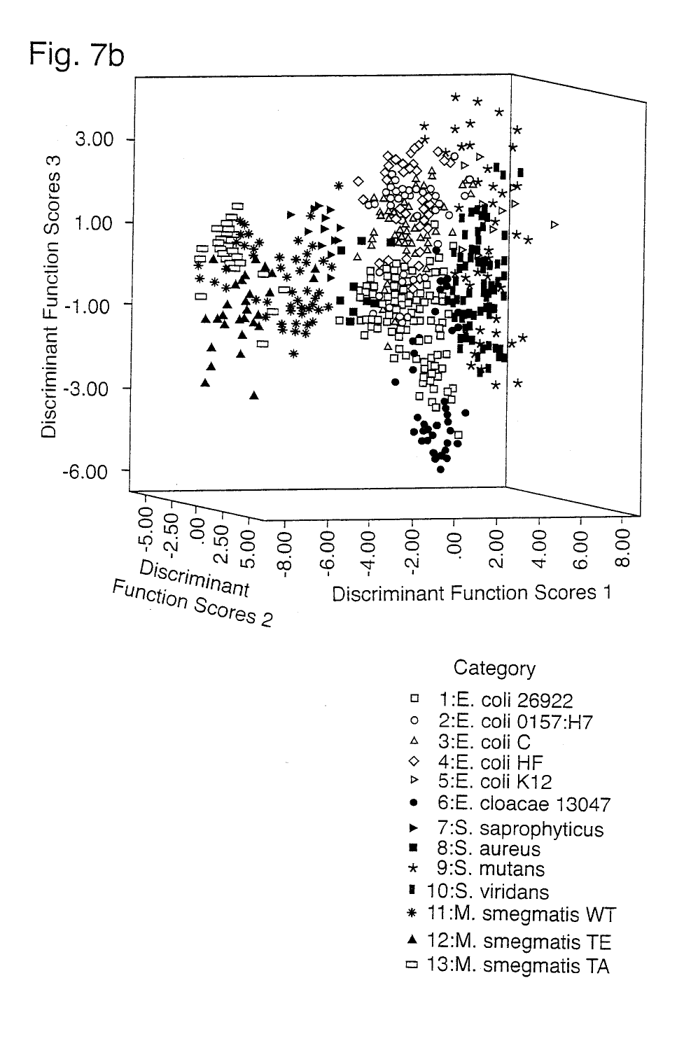

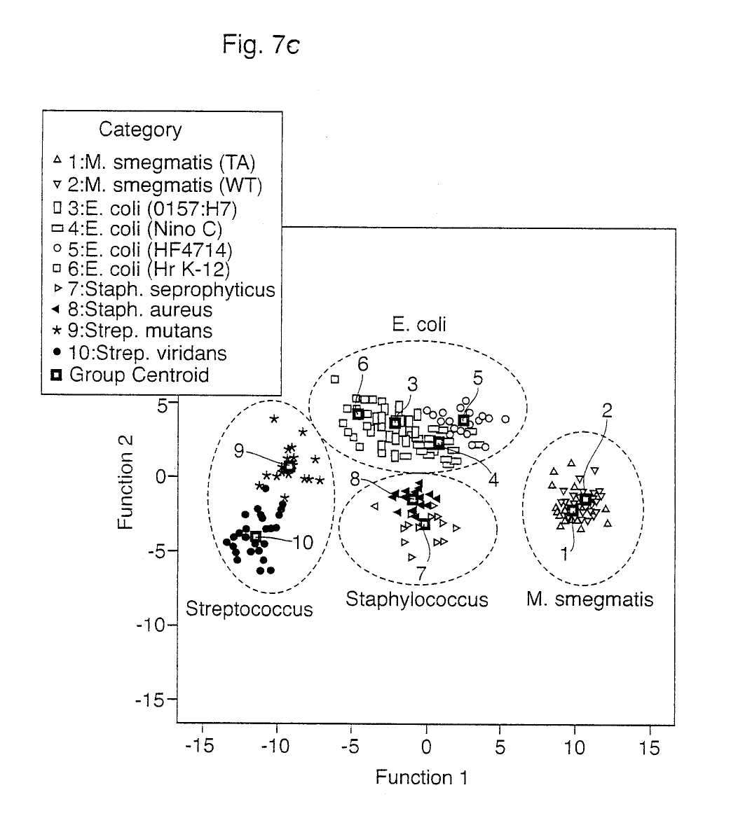

FIGS. 7a to 7c illustrate that bacteria can be discriminated/classified by genus or by species utilizing the LIBS spectrum. The results of a discriminant function analysis performed on over 600 spectra from 13 specimens of bacteria. The 13 specimens were comprised of bacteria representing five genera. Included in the analysis were two species of Staphylococcus (aureus and saprophyticus), two species of Streptococcus (mutans and viridans), five strains of E. coli, one strain of Enterobacter cloacae, and three strains of Mycobacterium smegmatis. Only the first two of 12 canonical discriminant functions are shown, as well as the “group centroids” or centers of mass of the clusters of points. The first three canonical discriminant functions are shown in a corresponding 3D plot (FIG. 7b) and the group centroids are removed for clarity. Table 3 shows truth tables for a genus level test showing the sensitivity (true positives) and specificity (false positives) of the identification.

TABLE 2

| | True | False |
|---|---|---|
| Escherichia | | |
| Positive | 89.97% | 4.28% |
| Negative | 95.72% | 10.03% |
| Staphylococcus | | |
| Positive | 62.16% | 2.55% |
| Negative | 97.45% | 37.84% |
| Streptococcus | | |
| Positive | 83.82% | 2.04% |
| Negative | 97.96% | 16.18% |
| Mycobacterium | | |
| Positive | 89.61% | 1.27% |
| Negative | 98.73% | 10.39% |

The results of a species/strain level identification test were as follows:

TABLE 3

| | | Predicted Group Membership (%) | | | | | | | | | |
|---|---|---|---|---|---|---|---|---|---|---|---|
| Group | | 1 | 2 | 3 | 4 | 5 | 6 | 7 | 8 | 9 | 10 |
| 1: | M. smegmatis (TA) | 82.4 | 17.6 | 0 | 0 | 0 | 0 | 0 | 0 | 0 | 0 |
| 2: | M. smegmatis (WT) | 28.0 | 72.0 | 0 | 0 | 0 | 0 | 0 | 0 | 0 | 0 |
| 3: | E. coli (0157:H7) | 0 | 0 | 96.0 | 4.0 | 0 | 0 | 0 | 0 | 0 | 0 |
| 4: | E. coli (Nino C) | 0 | 0 | 3.6 | 96.4 | 0 | 0 | 0 | 0 | 0 | 0 |
| 5: | E. coli (NF4714) | 0 | 0 | 0 | 0 | 100.0 | 0 | 0 | 0 | 0 | 0 |
| 6: | E. coli (HfrK-12) | 0 | 0 | 6.7 | 0 | 0 | 93.3 | 0 | 0 | 0 | 0 |
| 7: | Staph. saprophyticus | 0 | 0 | 0 | 0 | 0 | 0 | 94.1 | 5.9 | 0 | 0 |
| 8: | Staph. aureus | 0 | 0 | 0 | 0 | 0 | 0 | 0 | 100.0 | 0 | 0 |
| 9: | Strep. mutans | 0 | 0 | 0 | 0 | 0 | 0 | 0 | 0 | 95.0 | 5.0 |
| 10: | Strep. viridans | 0 | 0 | 0 | 0 | 0 | 0 | 0 | 0 | 0 | 100.0 |

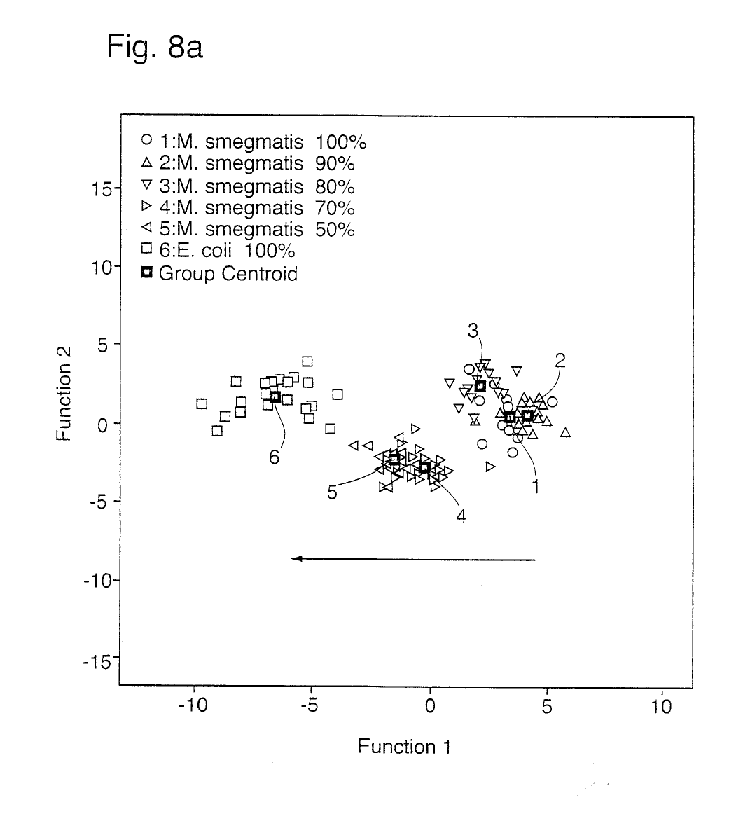

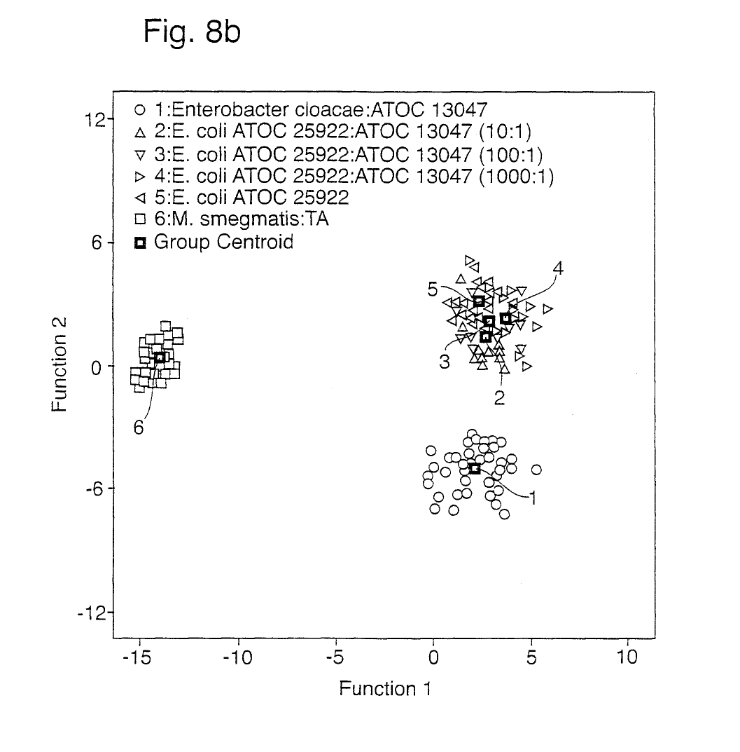

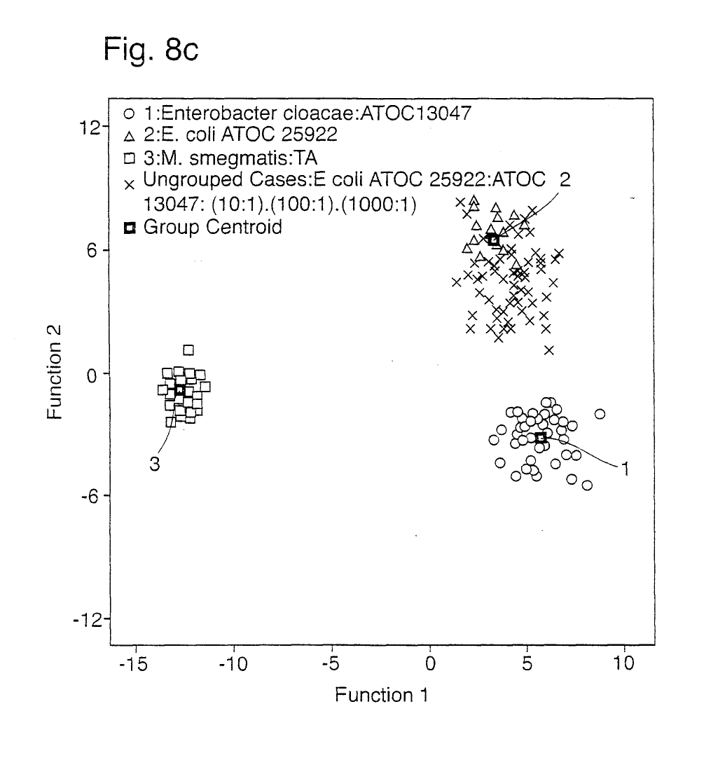

FIGS. 8a and 8b show that bacteria in mixtures (mixed samples) can be identified and the test is insensitive to bacterial contamination. Various mixtures of M. smegmatis and E. coli were prepared and the correct identification was achieved, with the greatest accuracy obtained where one species comprised 80% or more of the mixture (see Table 4). (FIG. 8a) E. coli and E. cloacae were mixed to represent common laboratory contamination levels (100:1) and (1000:1) (FIGS. 8b and 8c). At no time did the identification fail due to the presence of trace amounts of other cells.

TABLE 4

| | # of | Classification Results | | |
|---|---|---|---|---|
| Category | Spectra | M. smegmatis | E. coli | S. viridans |
| 100% M. smegmatis, | 21 | 100% | 0% | 0% |
| 0% E. coli | | | | |
| 90% M. smegmatis, | 20 | 100% | 0% | 0% |
| 10% E. coli | | | | |
| 80% M. smegmatis, | 16 | 100% | 0% | 0% |
| 20% E. coli | | | | |
| 70% M. smegmatis, | 21 | 76% | 34% | 0% |
| 40% E. coli | | | | |
| 50% M. smegmatis, | 19 | 47% | 53% | 0% |
| 50% E. coli | | | | |
| 0% M. smegmatis, | 25 | 0% | 100% | 0% |
| 100% E. coli | | | | |

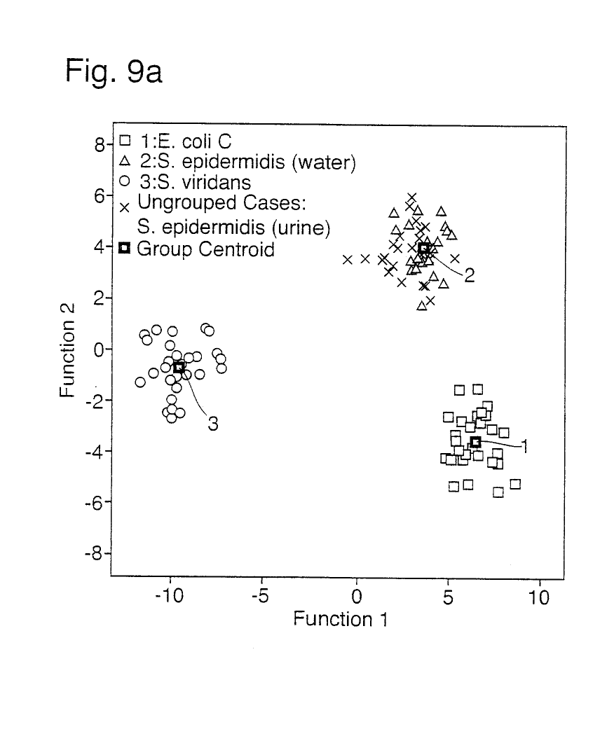

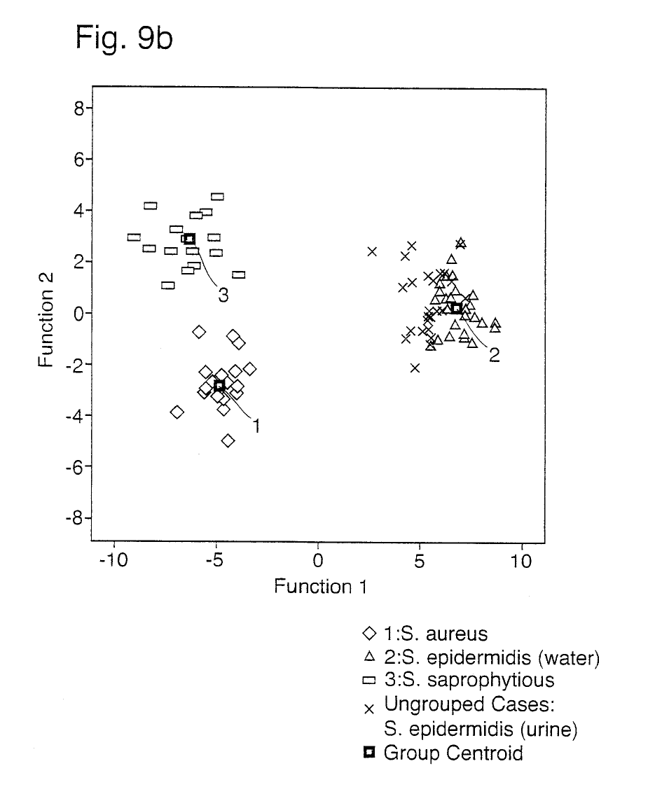

FIG. 9 shows that bacteria in sterile urine may be easily identified without washing or any other sample preparation. LIBS spectral fingerprints were obtained for Staph. epidermidis, E. coli, and S. viridans in a sterile water specimen. Spectra from S. epidermidis harvested directly from a spiked urine specimen were identified 100% as belonging to S. epidermidis. The test was repeated with three Staphylococci species (aureus, epidermidis, and saprophyticus) in sterile water specimens. Again, all urine-harvested S. epidermidis spectra were correctly identified as S. epidermidis.

The applicant has thus appreciated that the LIBS system 10 is operable in a variety of applications, including without limitation the rapid discrimination of bacterial cells from other biotypes (e.g. yeasts or molds), or the discrimination of the pathogenic enterohemmorhagic E. coli O157:H7 strain from other non-pathogenic E. coli strains. Again, it has been recognized that bacterial identification appears to be independent of the growth condition and culture medium in which the bacteria were grown (See FIG. 3a).

Bacterial LIBS spectra do not change with time as the bacterial population/colony ages on an abiotic surface (necessary for accurate identification and detection of surface contamination (See FIGS. 3a and 3b)). Further, experimental testing further suggests that bacterial LIBS spectra can be easily obtained from killed (via autoclaving) or inactivated (via UV light) specimens, and treatments which render specimens safe for handling do not decrease identification specificity, nor LIBS spectral intensity (See FIGS. 5a to 5c).

In addition, the intensity of the LIBS spectrum is linearly dependent on cell number, but the specificity is not dependent on cell number (See FIG. 6), and species of bacteria tested possess unique atomic compositions allowing a LIBS-based identification of unknown bacterial specimens (See FIGS. 7a to 7c).

Bacteria in mixed samples may further be identified, where for example the dominant or majority bacterial component of a two-component bacterial mixture is reliably identified, and preferably where it comprises at least 50%, and more preferably 80% or more of the mixture (See FIGS. 8a, 8b and 8c).

Further, with the present invention, bacteria can be identified when specimens are obtained from clinical samples (e.g. sterile urine containing organic and inorganic solutes) without the need to remove other compounds/molecules present in the specimen (See FIGS. 9a and 9b); and bacterial LIBS spectrum for a given species is stable and does not change with time (experiments conducted on the same E. coli strain over extended periods).

It is envisioned that the current invention has applications utilizing a variety of apparatus suitable to achieve LIBS analysis. These include, without limitation, dual-pulse laser systems, advanced signal-processing computerized routines, and femtosecond laser systems to lower the limits of detection for hazardous materials and to improve the range and flexibility of the systems.

The LIBS based system 10 requires no a priori genetic sequences or DNA primers, such as is required in RT-PCR, FISH, and DNA microarrays, nor extraction or amplification steps. In addition, LIBS based system 10 is comparatively inexpensive, designed to be usable by non-experts and is rapid and robust. This spectral signature data achieved with the present system can be analyzed and utilized on common laptop hardware with proprietary (IP) software. The LIBS system 10 does not require biochemical precursors or time sensitive consumables like antibody-based techniques, such as microarray technologies or high-throughput lab-on-a-chip assays or multi-well devices. In addition, the LIBS system 10 can identify a number of bacteria equally well, and mutations will not yield a “null” result.

The applicant has recognized that in general, LIBS is not as sensitive as techniques utilizing genetic amplification (where one segment of DNA is multiplied many-fold), as one trade-off for more increased speeds. However, in a preferred use, the target goal is to measure comparatively smaller numbers of cells (i.e. 100 cells, the infectious dose of pathogenic E. coli), which will make the LIBS system 10 clinically useful.

Because the detected LIBS spectrum of an undiagnosed bacterium is identified by matching it to a pre-compiled reference library, it may be difficult to identify organisms never previously encountered. Nonetheless, previously encountered pathogens will still be identified, likely as organisms closely resembling the same species or genus. In trial examples, species of Staphylococci not in the library were identified as Staphylococci on the basis of such similarities. This was also true for species of Streptococci. In an alternate mode, computerized identification based on the pattern matching algorithm may be operable to return a diagnosis with a “percent confidence value” that would indicate a correlation.

Proof-of-concept experiments suggest that the bacterial identification is adequately robust, has the requisite sensitivity and specificity, is impervious to contamination, is stable through time, and can be performed on bacteria from human specimens.

In an initial phase, the spectrally fingerprinting of hundreds of specimens from a few specially chosen target microorganisms are undertaken. Other organisms can of course be easily added as desired, depending on the target bacteria. The spectral fingerprints are preferably obtained from pure cultures of known, well-defined strains grown in the microbiology lab. The sensitivity, specificity, and reproducibility of the identification is verified via blind testing of the spectral library with “unknown” samples prepared in the same way. Again, acceptable computerized identification algorithms are preferably used. Optionally, identical tests are performed on the MALDI-TOF instrument for comparison.

The applicant envisions a number of possible methods of sample collection and/or preparation for LIBS analysis using the system 10. These include filtering of liquid specimens with common microbial (e.g. 0.22 or 0.45 μm) filter paper and LIBS sampling on that filter; centrifugation with filtered centrifuge tubes; differential centrifugation; investigation of flow-through microfluidic separation utilizing hydrodynamic microfluidic separation or optical/laser separation; dielectrophoresis; and phage display technology/antibody-tagging for bacterial cell fixing.

In a most preferred embodiment, the method described above is used to test and determine antibiotic resistance. In such a test the bacteria obtained via any of the methods described above is tested for evidence of resistance to common antibiotics by the steps of:
1. Concentrating bacteria in a specimen to a sufficient titer, and dividing the specimen so obtained into a number of equal test fractions;
2. Optionally placing one of the test fractions so created into a control liquid solution i.e. (water, or PBS) where growth is not possible;
3. Placing other such test fractions in a known pre-selected liquid growth medium or broth, where bacterial growth is expected;
4. Placing further such test fractions in known pre-selected liquid growth mediums or broths containing antibiotics or antimicrobial agents;
5. Placing all specimen test fractions in an environment conducive to bacterial growth (including proper temperature, humidity, gas atmosphere, etc.) for a selected period of time as for example which is guaranteed to allow for cell division to any arbitrary number, such as a doubling or tripling of the expected cell number;
6. Removing of the specimen test fractions following the selected period of time and optionally further separating and/or concentrating as described hereafter;
7. Following removal and bacterial preparation, testing of the bacterial specimens from all liquid samples using the system 10 and LIBS method described above is undertaken. The results obtained specifying a control number of baseline bacteria (the sample in non-growth medium establishing a baseline cell count which will be indicated by the total LIBS emission intensity in the spectrum); a control number of growing bacteria (the sample in the growth medium establishing an elevated number of cell dues to reproductive activity which will be indicated by a total LIBS emission intensity in the spectrum much greater than was observed in the baseline control); and a test number of bacteria (the sample placed in the antibiotic containing medium is the test sample—if it is antibiotic sensitive, the cell number will not have increased, and the measured LIBS emission intensity will be equal to or less than the intensity measured in the baseline control. If the bacteria are antibiotic resistant, the cell number will have increased and the measured LIBS emission intensity will be larger than the baseline control, and may be equal to the growing bacteria control or a little less), and
8. The results of the LIBS emission intensities are measured with the baseline control intensity, the growth control intensity, and the test intensity output reported; and
9. Antibiotic resistance of the test samples is thus interpreted by way of the comparison of the test samples total LIBS emission intensity to the total LIBS emission intensities of the two controls.
   Optionally, the method may further include isolating/collecting the bacteria prior to LIBS testing by the steps of:
1. A liquid specimen is prepared via use methods such that the bacteria are separated from other components in suspension;
2. Microfluidic devices are used that separate cells in a flow via one or more of cell mass, cell size, thermodynamic radius, or any combination thereof;
3. Microfluidic devices are used that separate cells via optical trapping or laser based methods;
4. Microchannel devices are used that separate cells via integrated dielectrophoresis components; and/or
5. Microchannel or open channel flow devices are used that separate and collect cells in specific locations via bimolecular bonding to phages, or antibodies, or functionalized thiol groups, or any other immunochemical treated molecule.

The applicant has appreciated that fewer tests may be administered due to the presence of an accurate complementary diagnostic test like the LIBS instrument proposed herein, time may be reduced. The primary advantage of the use of LIBS technology is speed, as it is anticipated to be possible to identify a pathogen in less than 5 minutes, whereas it will take 24-72 hours for the current technologies to identify an unknown pathogen. The expeditious result of testing is advantageous to physician/care-provider and patient, as rapid detection is important for health and safety reasons, more so if the diseases are highly contagious, and the ability to identify the pathogen in an accurate and fast manner may prove crucial in preventing an outbreak of a contagious diseases.

The present invention thus provides in a most preferred aspect a technology that can achieve rapid and independent verification of bacterial identity that does not require a priori knowledge of nucleic acid sequences or antibodies against known bacterial antigens. The LIBS system further may be operated to identify pathogenic bacteria in clinical samples at “time zero” (the time when a specimen of blood, urine, or sputum is obtained) with minimal or no sample preparation required.

While the exemplary embodiments provided describe the operability of the present invention as used for confirming the presence of E. coli and Staphylococci bacterial strains, the invention is not so limited. The present apparatus may be used to identify and/or confirm the presence of a variety of different pathogens. In one alternate preferred use, the LIBS system 10 may be used to identify and/or confirm the presence of clostridium difficile (c. difficile) bacteria in nursing homes and medical facilities.

While the detailed description describes various preferred aspects, the invention is not so limited. Many modifications and variations will occur to persons skilled in the art. For a definition of the invention, reference may be had to the appended claims.

We claim:

1. A pathogen detection system comprising,
a specimen support for supporting a test specimen sample to be analyzed,
a coherent light source operable to direct a coherent light beam at said specimen support to at least partially atomize said test specimen sample,
an optical detector positioned for detecting a spectral signature of electromagnetic radiation emitted, reflected, or absorbed by the at least partial atomization of the test specimen sample,
an analyzer for comparing the detected spectral signature to at least one predetermined spectral signature for one or more pathogens.

2. The system as claimed in 1, wherein the specimen sample is selected from the group consisting of a bacterial culture, a tissue biopsy and a body fluid specimen.

3. The system as claimed in claim 1 or claim 2, wherein the pathogen comprises a bacteria.

4. The system as claimed in claim 3, wherein the bacteria is selected from the group consisting of Escherichia, Staphylococcus, S. viridans, S. epidermidis, E. cloacae, M. smegmatis Bacillus anthracis and c. difficile.

5. The system as claimed in any one of claims 1 to 4, wherein said coherent light beam comprises a pulsed laser beam.

6. The system as claimed in any one of claims 1 to 5, wherein the test specimen sample comprises one of a plurality of test samples prepared from a sample.

7. The system as claimed in claim 6, wherein the analyzer is further operable to compare an intensity of the detected spectral signature of the test specimen sample with an intensity of a stored spectral signature of at least one other of said test samples.

8. The system as claimed in any one of claims 1 to 7, wherein said coherent light beam has a focused beam diameter at said specimen support selected at less than about 250 μm, and preferably at about 100 μm.

9. The system as claimed in any one of claims 1 to 8, wherein said test specimen sample comprises bacteria cells concentrated to at least about 50% by cell-count titer, and preferably at least 70% by cell-count titer.

10. A method of detecting a pathogen using the pathogen detection system of any one of claims 1 to 9, comprising the steps of,
providing said test specimen sample on said specimen support,
actuating said coherent light source to generate said coherent light beam to at least partially atomize said test specimen sample to effect the emission reflection and/or absorption of electromagnetic radiation with said optical detector, collecting and storing a detected spectral signature of said electromagnetic radiation, and
comparing the detected spectral signature of said test specimen sample with one or more of said predetermined spectral signatures.

11. The method as claimed in claim 10 further comprising a step of concentrating said pathogen content in a sample, and dividing said sample into a plurality of substantially equally sized fractions, and selecting one of said fractions as said test specimen sample.

12. The method of claim 10 or claim 11, wherein said pathogen comprises bacteria, and further comprising concentrating said bacteria to a concentration of at least about 70% by cell-count titer, and preferably at least about 80% by cell-count titer, prior to providing said pathogen in said test specimen sample.

13. The method of any one of claims 10 to 12, wherein said test specimen sample comprises a culture media selected from the group consisting of TSA, MAC and deoxycholate-spiked agar.

14. The method of any one of claims 10 to 13, wherein said specimen support comprises a purified agar support having between about 1 and 2% by wt solid culture media.

15. The method of any one of claims 10 to 13, wherein said coherent light beam is activated to produce a pulsed beam having a beam diameter at said test specimen sample of between 50 μm and 150 μm.

16. The method of any one of claims 10 to 15, wherein said analyzer compares the detected spectral signature of the test specimen sample with at least one of a detected or predetermined spectral signature of the specimen support.

17. A method of determining pathogen drug or antibiotic resistance using a laser-induced breakdown spectroscopy system comprising,
a specimen support for supporting test specimen samples to be analyzed thereon,
a coherent light source operable to direct a coherent light beam at said specimen support to at least partially atomize said test specimen samples,
an optical detector positioned for detecting the spectral signatures of electromagnetic radiation emitted by the at least partial atomization of a selected test specimen sample, and
an analyzer operable to compare the detected spectral signature to at least one stored spectral signature of another specimen sample, said method comprising the steps of:
providing a first test specimen sample representative of an untreated, or drug or antibody treated sample at a first period of time,
with said first test specimen sample on said specimen support, actuating said coherent light source to at least partially atomize said first test specimen sample,
with said optical detector, detecting a spectral signature of electromagnetic radiation emitted by the at least partial atomization of said first test specimen sample as one said stored spectral signature,
providing a second test specimen sample representative of the drug or antibody treated sample at a second period of time,
with said second test specimen sample on said specimen support, actuating said coherent light source to at least partially atomize said second test specimen sample,
with said optical detector, detecting the spectral signature of electromagnetic radiation emitted by the at least partial atomization of said second test specimen sample, and
with said analyzer, comparing an intensity of the spectral signature of the second test specimen sample with an intensity of the stored spectral signature.

18. The method as claimed in claim 17, further comprising outputting an increase in the intensity of spectral signature of the second test specimen sample as an indication of an increase in pathogen resistance to said drug or antibody.

19. The method as claimed in claim 17 or claim 18, wherein the pathogen comprises a bacteria.

20. The method as claimed in claim 19, wherein the bacteria is selected from the group consisting of Escherichia, Staphylococcus, S. viridans, S. epidermidis, E. cloacae, M. smegmatis Bacillus anthracis and c. difficile.

21. The method of any one of claims 17 to 20, comprising an earlier step of concentrating said pathogen content in a sample, and preferably concentrated to at least about 70% by cell-count titer, and dividing said sample into a plurality of substantially equally sized fractions, and selecting individual ones of said fractions as said first and second test specimen sample.

22. The method as claimed in any one of claims 17 to 21, wherein said test specimen sample comprises a culture media selected from the group consisting of TSA, MAC and deoxycholate-spiked agar.

23. The method as claimed in any one of claims 17 to 21, wherein said specimen support comprises a purified agar support having between about 1 and 2% by wt solid culture media.

24. The method as claimed in claim 23, said analyzer compares the detected spectral signature of the test specimen sample with at least one of a detected or predetermined spectral signature of the specimen support.
